(19) United States
(12) Patent Application Publication
(10) Pub. No.: US 2005/0010125 A1
(43) Pub. Date: Jan. 13, 2005

(54) Systems and methods for respiration measurement

(76) Inventors: James A. Joy, Alpharetta, GA (US); Wilton J. Pyle III, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/721,476

(22) Filed: Nov. 25, 2003

Related U.S. Application Data
(60) Provisional application No. 60/429,109, filed on Nov. 26, 2002.

(51) Int. Cl. A61B 5/08

(52) U.S. Cl. 600/529

(57) ABSTRACT

In one embodiment, a portable data acquisition unit includes a pressure sensor that is configured to measure pressure signals at a high-frequency rate, the signals pertaining to patient breathing, a microcontroller that receives pressure signals measured by the pressure sensor and determines clocks times associated with the pressure signals, and an interface that is configured to output sleep session data from the data acquisition unit to another device.

### CROSS-REFERENCE TO RELATED APPLICATION

This application claims priority to copending U.S. provisional application entitled, “System and Method for Respiration Measurement,” having Ser. No. 60/429,109, filed Nov. 26, 2002, which is entirely incorporated herein by reference.

### BACKGROUND

Sleep disordered breathing (SDB) is a serious, potentially life-threatening condition that is far more common than generally understood. A major component of the SDB spectrum is obstructive sleep apnea syndrome (OSAS), commonly referred to as sleep apnea. Sleep apnea is a breathing disorder characterized by brief interruptions of breathing during sleep. The two types of sleep apnea are central and obstructive. Central sleep apnea, which is less common, occurs when the brain fails to send the appropriate signals to the breathing muscles to initiate respiration. Obstructive sleep apnea is far more common and occurs when air cannot flow into or out of the person's nose or mouth although efforts to breathe continue. Other possible pathologies of SDB include hypopneas, respiratory effort related arousals (RERAs), upper airway resistance syndrome (UARS), Cheyne-Stokes breathing, and snoring.

In a given night, the number of breathing pauses, or apneas, may be as high as 20 to 30 or more per hour. These apneas are typically accompanied by snoring between apnea episodes, although not everyone who snores has this condition. Sleep apnea can also be characterized by choking sensations. The frequent interruptions of deep, restorative sleep often lead to early morning headaches and excessive daytime sleepiness. Early recognition and treatment of sleep apnea is important because it may be associated with irregular heartbeat, high blood pressure, heart attack, stroke, and even death.

Currently, approximately 95 percent of people who have sleep apnea do not realize they have it. There are about 19 million undiagnosed people with sleep apnea in the United States. Therefore, greater efforts must be made to diagnose this condition. Sleep apnea presently is diagnosed in sleep laboratories in which the patient's breathing patterns during sleep are monitored and interpreted. At such laboratories, nasal and oral airflow is measured, in addition to up to 12 to 14 additional channels of information, so as to obtain a complete measurement of full respiration. Unfortunately, however, laboratory diagnoses are costly and inconvenient to the patient.

Due to the high cost and inconvenience of laboratory diagnosis, a number of home screening and diagnostic devices have been developed. These screening and diagnostic devices unfortunately lack the reliability, sensitivity, and specificity that is needed to obtain results comparable to those that may be obtained in a sleep laboratory. Accordingly, it would be desirable to have a system and method for measuring respiration that is both portable so that it can be used at the patient's home and reliable and sensitive enough for effective screening and diagnosing of sleep apnea and other SDBs.

### BRIEF DESCRIPTION OF THE DRAWINGS

The disclosed systems and methods can be better understood with reference to the following drawings. Features shown in the drawings are not necessarily to scale.

FIG. 1 is a schematic view of an embodiment of a system for measuring respiration, shown used in conjunction with a patient.

FIG. 2 is a block diagram of an embodiment of a data acquisition unit of the system shown in FIG. 1.

FIG. 3 is a block diagram of an embodiment of memory of the data acquisition unit shown in FIG. 2.

FIG. 4 is a flow diagram that illustrates an embodiment of operation of the system of FIG. 1.

FIGS. 5A and 5B provide an embodiment of operation of the data acquisition program shown in FIG. 3.

FIGS. 6-11 are plots of breathing pressure versus time recorded by the data acquisition unit of FIG. 2.

FIG. 12 illustrates an example of downloading data from the data acquisition unit of FIG. 1 to a computer.

FIG. 13 is a block diagram of an embodiment of the computer shown in FIG. 12.

FIGS. 14-22 illustrate example interface screens that can be presented to a user by the data analysis program shown in FIG. 13.

### DETAILED DESCRIPTION

As described above, needed is a system and method for measuring respiration at home so that sleep disordered breathing (SDB) can be screened and/or diagnosed. If accurate results could be obtained with such a system and method, SDB could be discovered much less expensively and with greater convenience for the patient. Disclosed herein are such systems and methods. As is described below, the systems include a comfortable patient interface and a data acquisition unit that is capable of capturing data regarding respiratory flow. In some cases, the data acquisition unit not only records respiration data, but also analyzes the data to make SBD diagnoses. Through use of the systems, diagnosable are, for example, sleep apnea, hypopneas, respiratory effort related arousals (RERAs), upper airway resistance syndrome (UARS), Cheyne-Stokes breathing, and snoring.

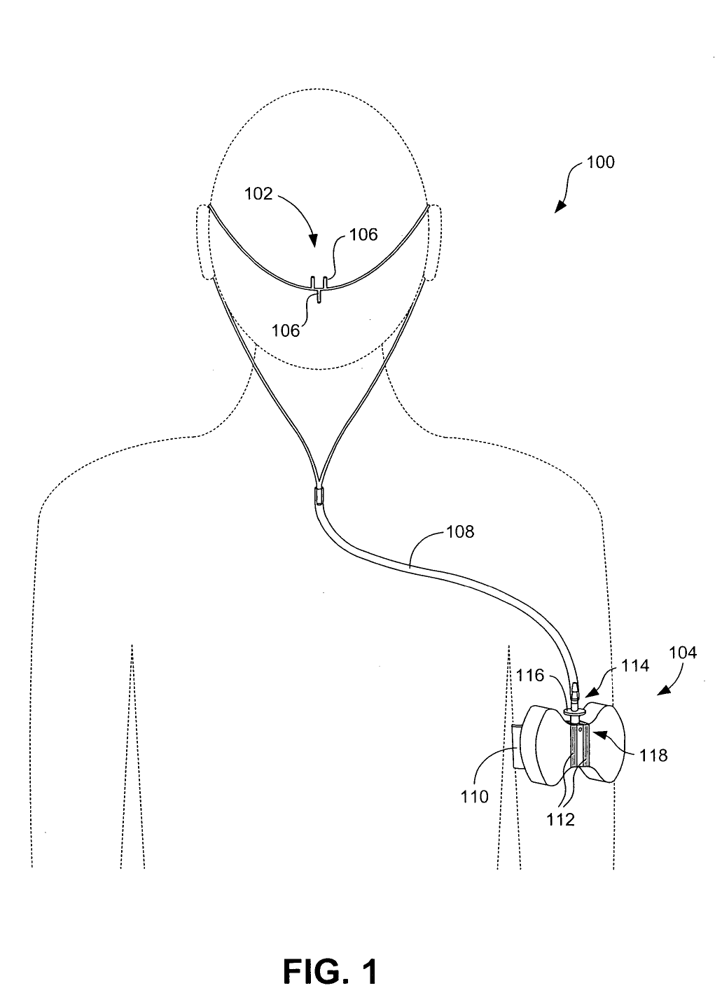

Referring now to the drawings in which like numerals identify corresponding parts, FIG. 1 illustrates an embodiment of a system 100 for measuring respiration. As indicated in FIG. 1, the system 100 generally comprises a patient interface 102 that is placed adjacent the patient's nose and mouth and a data acquisition unit 104 that, for example, is worn by the patient while sleeping. The patient interface 102 can take many different forms. In the embodiment shown in FIG. 1, the interface 102 is formed as a cannula that passes between the patient's nose and mouth. In such an embodiment, the cannula may be provided with extension tubes 106 that extend outwardly toward the patient's nostrils and/or mouth. Alternatively, the cannula may instead merely include openings (not shown) that are directed at the patient's nostrils and/or mouth. In another embodiment, the patient interface can comprise a mask (not shown) that fits around one or both of the patient's nose and mouth. Although use of a mask may provide advantages over the cannula interface, a mask may be less desirable from a patient comfort standpoint. In any case, the patient interface 102 is preferably disposable so that it may be used once by the patient and then discarded.

Irrespective of the configuration of the patient interface 102, the interface connects to the data acquisition unit 104 with connector tube 108. As is indicated in FIG. 1, the data acquisition unit 104 may be worn by the patient around the patient's arm. By way of example, the data acquisition unit 104 attaches to the arm with an arm band 110 that wraps around the upper arm with the unit being attached to the arm band with elastic straps 112. Alternative placement of the data acquisition unit 104 is possible. For example, the unit 104 may include a clip (not shown) that permits the unit to be attached to a patient garment such as pajama pants, shorts, or a shirt. Alternatively, the data acquisition unit 104 may comprise a chest or waist band (not shown) so that the unit may be worn around the chest or waist. As is further shown in FIG. 1, the data acquisition unit 104 connects to the tube 108 with a port tube 114. Optionally, the port tube 114 includes an integral bacterial and/or hydrophobic filter 116 that filters bacteria and/or water before it reaches the data acquisition unit 104 so that it does not contaminate the unit. In addition to the aforementioned components, the data acquisition unit 104 may optionally include an indicator 118, such as a light-emitting diode (LED), that signals to the user when the device is activated.

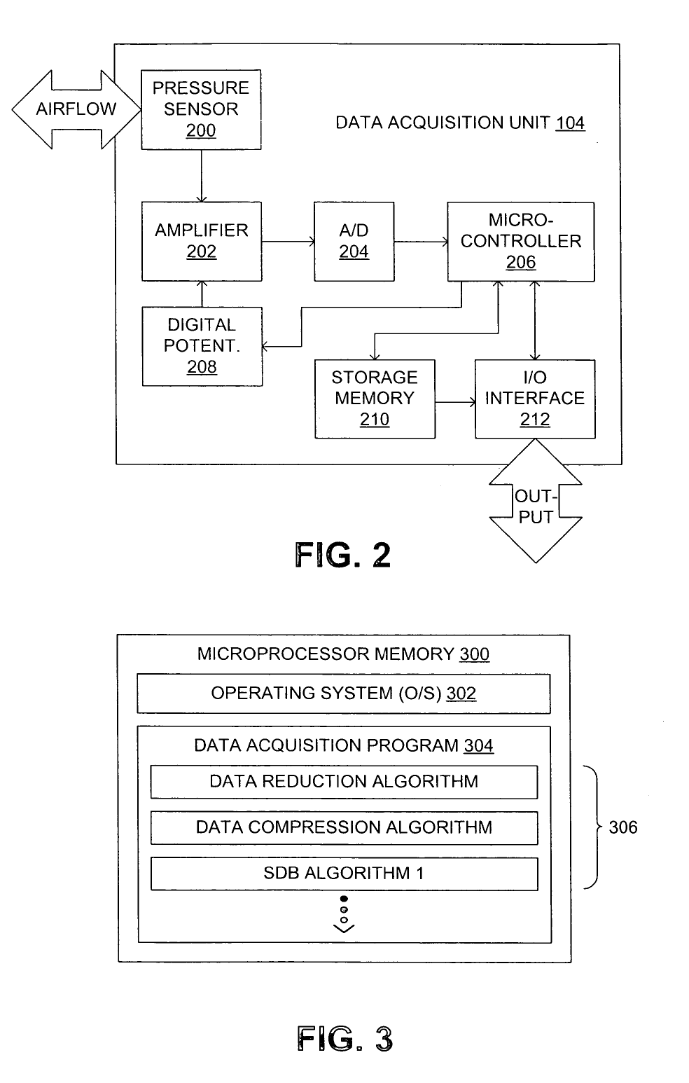

FIG. 2 illustrates an example architecture of the data acquisition unit 104. As indicated in this figure, the data acquisition unit 104 comprises a pressure sensor 200, an amplifier 202, an analog-to-digital (A/D) converter 204, a microcontroller 206, a digital potentiometer 208, storage memory 210, and an input/output (I/O) interface 212. The pressure sensor 200 may comprise a solid-state pressure sensor that includes a plurality of strain gauges that measure pressure fluctuations caused by patient respiration. Normally, the pressure fluctuations are measured in relation to a reference pressure (i.e., gauge pressure). The sensor 200 is tuned to measure relatively low-pressures to optimize the amount of signal obtained relative to noise. The pressure sensor 200 then outputs electrical differential output to the amplifier 202. By way of example, the pressure sensor 200 comprises a differential bridge, true low-pressure, silicon die sensor having model designation SM5455-001-G manufactured by Silicon Microstructures, Inc.

The analog pressure signals output from the pressure sensor 200 are amplified by the amplifier 202 prior to being converted into a digital signal by the A/D converter 204. In particular, the signal from the pressure sensor 200 drives an amplifier stage that performs scaling and shifting and provides anti-aliasing by limiting the bandwidth of the output signal. After the signal is converted into a digital signal, it is provided to the microcontroller 206. The microcontroller 206 comprises, for example, a digital signal processor (DSP) that includes internal non-volatile memory (e.g., Flash memory) that stores various firmware that controls data acquisition unit operation and, optionally, analyzes the raw data received from the pressure sensor 200 prior to being stored in the storage memory 210. The nature of this firmware is discussed below with regard to FIG. 3. The microcontroller 206 also controls the digital potentiometer 208 so that the amplification provided by the amplifier 202 can be adjusted. For instance, the gain of the amplifier 202 may be increased for relatively-light breathers, or decreased for relatively-heavy breathers. This gain is adjusted in relation to the reference pressure stored by the microcontroller 206. By way of example, the microcontroller 206 comprises a model MSP430F1491PM ultralow-power microcontroller manufactured by Texas Instruments, which has a 16-bit RISC architecture core with 16-bit CPU-integrated registers and a constant generator. Optionally, the microcontroller 206 may comprise the A/D converter 204. Regardless, the microcontroller 206 includes one or more internal clocks that is/are used as a means to time the acquisition session (i.e., a session timer).

The storage memory 210 is used to store data collected by the data acquisition unit 104 and preferably comprises non-volatile memory (e.g., Flash memory). The memory can have a capacity of, for example, approximately 8 megabytes (MB). As is described in greater detail below, the storage memory 210 can be used to store relatively low-frequency sampled signals (e.g., 10 samples per second) or relatively high-frequency sampled signals (e.g., 6000-10,000 samples per second) depending upon the information that is desired. In addition or in exception, data analysis may be stored within the storage memory 210. By way of example, the storage memory 210 can comprise a 64 Mbit, non-volatile, low-power consumption memory having model designation LHF64F12 manufactured by Sharp Electronics.

Each of the above components is powered by an on board power supply such as a battery (not shown). For example, a 3 volt (V) N-size lithium battery may be used. In order to reduce power consumption, each of the data acquisition unit components are preferably selected so as to require relatively little power during operation.

Once data are stored in the storage memory 210, these data may be transmitted to another device (e.g., personal computer (PC)) for further analysis or output as a report. Such transmission is facilitated with the I/O interface 212 that, for example, comprises a universal serial bus (USB) interface. Once the data are transmitted to the other device, they may, optionally, be stored in a central data repository for further analysis.

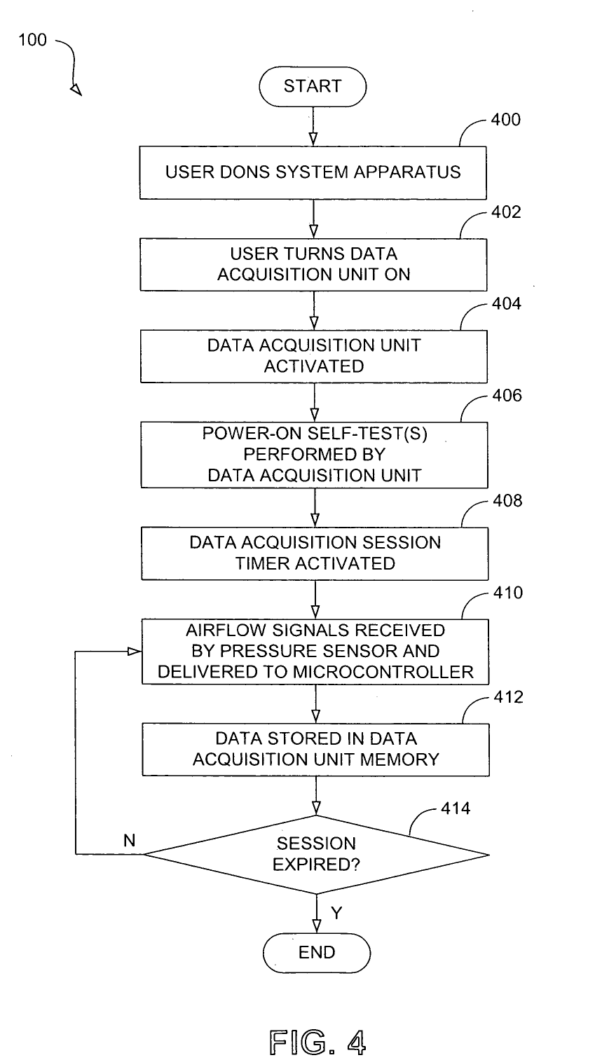

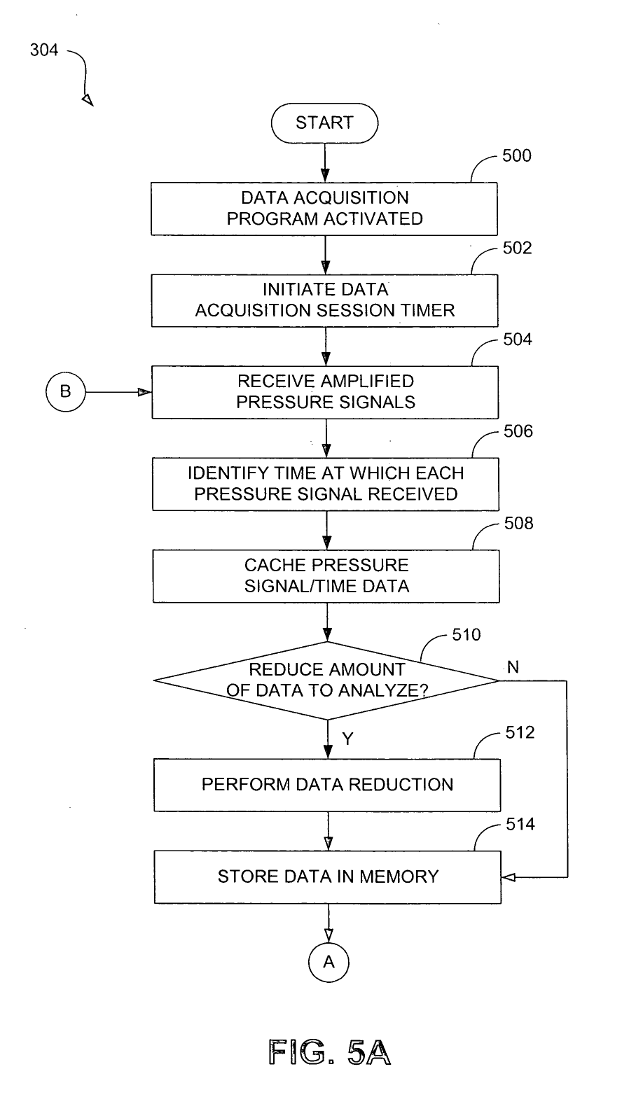

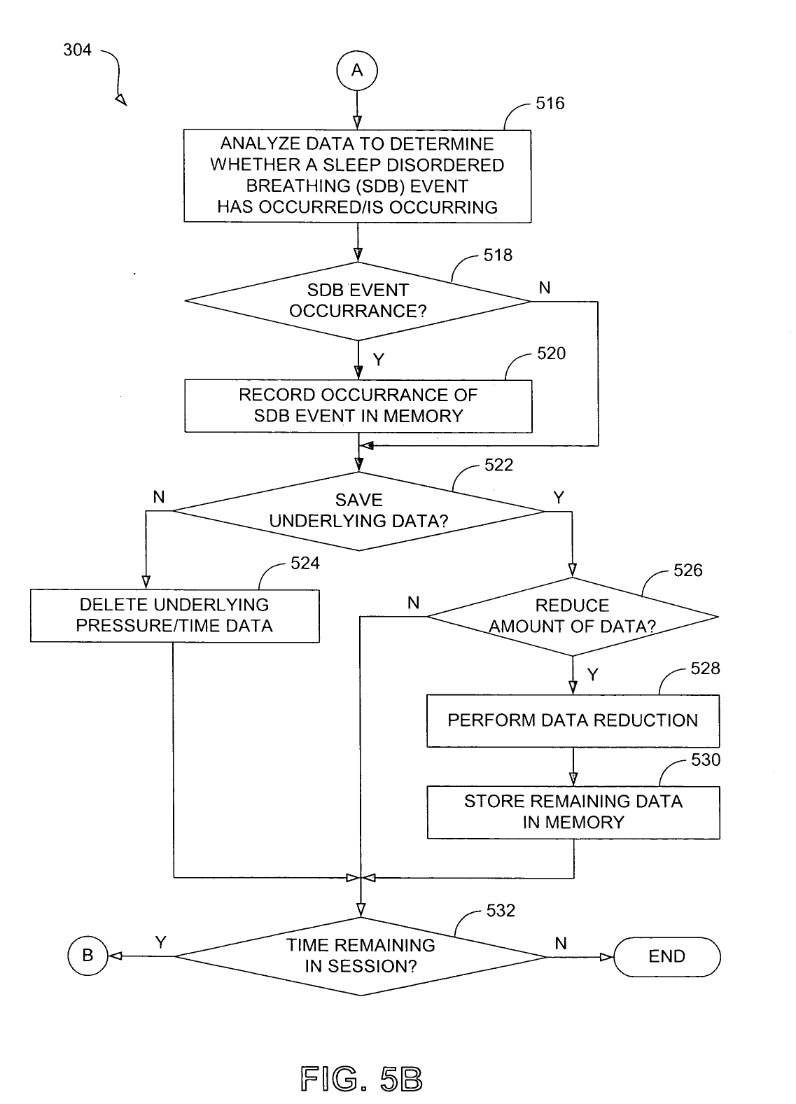

FIG. 3 illustrates an embodiment of memory 300 of the data acquisition unit 104 and, in particular, the microcontroller 206. As shown in this figure, the microcontroller 206 comprises, in firmware, an operating system (O/S) 302 that controls the general operation of the data acquisition unit 104, and a data acquisition program 304 that is used to collect and/or evaluate the signals received by the pressure sensor 200. As indicated in the figure, the program 304 may comprise various algorithms 306 that are used in the data acquisition/analysis performed by the data acquisition unit 104. For example, the program 304 may comprise a data reduction algorithm that is used to reduce the number of data to be analyzed or stored. In addition, the program 304 may comprise various sleep disordered breathing (SDB) analysis algorithms that interpret the collected data (raw or reduced) and make determinations as to SDB events. Furthermore, the program 304 can include a compression algorithm with which the data can be compressed so as to reduce the capacity requirements of the storage memory 210. The operation of the data acquisition program 304 of the microcontroller 206 is explained in greater detail below in relation to FIGS. 4 and 5. Although the program 304 has been shown and described as being provided within the microcontroller 206, it could, alternatively, be provided in a separate memory (e.g., storage memory 210) if desired.

An example system 100 having been described above, an example of operation of the system will now be discussed with respect to the flow diagram of FIG. 4. Any process steps or blocks in flow diagram of this disclosure may represent modules, segments, or portions of code that include one or more executable instructions for implementing specific logical functions or steps in the process. Although particular example steps are described, alternative implementations are feasible. Moreover, steps may be executed out of order from that shown or discussed, including substantially concurrently or in reverse order, depending on the functionality involved.

The apparatus of the system 100 can be obtained by a patient from a prescribing physician where sleep disordered breathing is suspected by the physician. In particular, the physician can provide a data acquisition unit 104 to the user, as well as a patient interface 102. As noted above, the patient interface 102, irrespective of its form, normally is disposable such that the user may discard it after use.

Once the apparatus is received, the patient may take the apparatus home to be used at night to record information about the patient's breathing patterns during sleep and, potentially, diagnose sleep disordered breathing. Accordingly, with reference to block 400 of FIG. 4, the patient (i.e., user) dons the system apparatus. The manner in which the apparatus is donned depends upon its configuration. In the system embodiment shown in FIG. 1, for example, donning may comprise attaching the data acquisition unit 104 to the user's arm and positioning the user interface 102 in place about the user's face such that the extension tubes 106 extend toward the nostrils and/or the mouth.

After the apparatus has been donned and the user gets into bed, the user then turns the data acquisition unit 104 on, as indicated in block 402. This can be accomplished, for instance, by manipulating a control element (e.g., button or switch) provided on the unit (not shown) or by pulling a removable activation tab (not shown). Through this action, the data acquisition unit 104 is activated, as indicated in block 404. Once activated, the unit 104 performs one or more power-on self-tests (POSTs) to ensure that the unit is operating properly, as indicated in block 406. Assuming the unit 104 to be in proper working order, the unit session timer is activated, as indicated in block 408, and, as indicated in block 410, airflow signals generated by the patient's breathing (captured by the user interface 102) are received by the pressure sensor 200. As is described in greater detail below with reference to FIGS. 5A and 5B, these signals may be stored into memory 210, analyzed to make sleep disordered breathing determinations, or both. In any case, however, data are stored in data acquisition unit memory 210, as indicated in block 412. It is because these data are stored locally within the unit memory 210 that portability of the system 100 is facilitated.

As data are stored, it is continually determined whether the data acquisition session has expired, as indicated in decision block 414. By way of example, the duration of the session may be set to be approximately eight hours, reflective of the duration of a typical night's sleep. Other durations could, of course, be used. If the predetermined session time has not expired, flow continues back to block 410 at which signals are continued to be received, and to block 412 at which data are stored. Once the session time has expired, however, flow is terminated. At this point, the data acquisition unit 104 may be returned to the prescribing physician and the data stored thereon downloaded to a computer (e.g., PC) or directly to a printer for the purpose of producing a hard copy report for the physician.

FIGS. 5A and 5B illustrate an example of operation of the data acquisition program 304 of the data acquisition unit 104. Beginning with block 500 of FIG. 5A, the data acquisition program 304 is activated, for instance when the user turns the data acquisition unit 104 on. Once the data acquisition program 304 is activated, the program initiates the data acquisition session timer, as indicated in block 502, and receives amplified pressure signals, as indicated in block 504. As noted above, these signals may, for example, be obtained with a high frequency (e.g., 6000-10,000 samples per second). Such high-frequency collection of pressure signals facilitates the diagnosis of sleep disordered breathing that may not otherwise be detected if lower-frequency collection is used. For instance, snoring, which may be indicative of sleep disordered breathing, may be detected where high-frequency data collection is used. The signals comprise digital signals that have been converted from analog signals by the A/D converter 204. As each signal is received, the time at which the signal is received is identified by the program 304, as indicated in block 506, for purposes of recording breathing patterns as a function of time and/or to facilitate determinations as to whether sleep disordered breathing events are occurring. Once the signal data and time data are possessed, these data are cached, for instance in microprocessor memory, as indicated in block 508, for temporary storage.

With reference to decision block 510, the program 304 determines whether to reduce the total amount of data that is to be analyzed. In particular, analysis can be conducted on all received data, or a portion thereof. Where the signal frequency is particularly high and the events to be detected may be detected with a relatively low frequency of signals, it may be desirable to reduce the number of data. If, on the other hand, a high frequency of signals is believed necessary or beneficial to the detection of a given event, it may be desirable to analyze all such signals. In the latter case, flow continues down to block 514 described below. If, however, it is determined in decision block 510 to reduce the amount of data to analyze (e.g., determined in view of a physician-selected mode), flow continues to block 512 at which the program 304 performs some form of data reduction on the data. By way of example, data decimation can be performed by a data reduction (e.g., decimation) algorithm of the data acquisition program 304 and may comprise the discarding of or selection of given received signals. For instance, decimation may comprise selection of every fifth signal and discarding of the remainder. Alternatively, decimation may comprise averaging groups of signals (e.g., groups of 10) to produce a series of averaged signals.

Whether data reduction is performed or not, data are stored in data acquisition unit memory, as indicated in block 514. Referring now to FIG. 5B, it is assumed that the data acquisition unit 104, and the program 304 in particular, will analyze the collected data. Accordingly, with reference to block 516 of FIG. 5B, the stored data are analyzed by the data acquisition program 304 to determine whether a sleep disordered breathing event has occurred or is occurring. In particular, one or more sleep disordered breathing (SDB) algorithms of the data acquisition program 304 are used to analyze the collected data. Example algorithms are identified in the following.

Apnea Detection

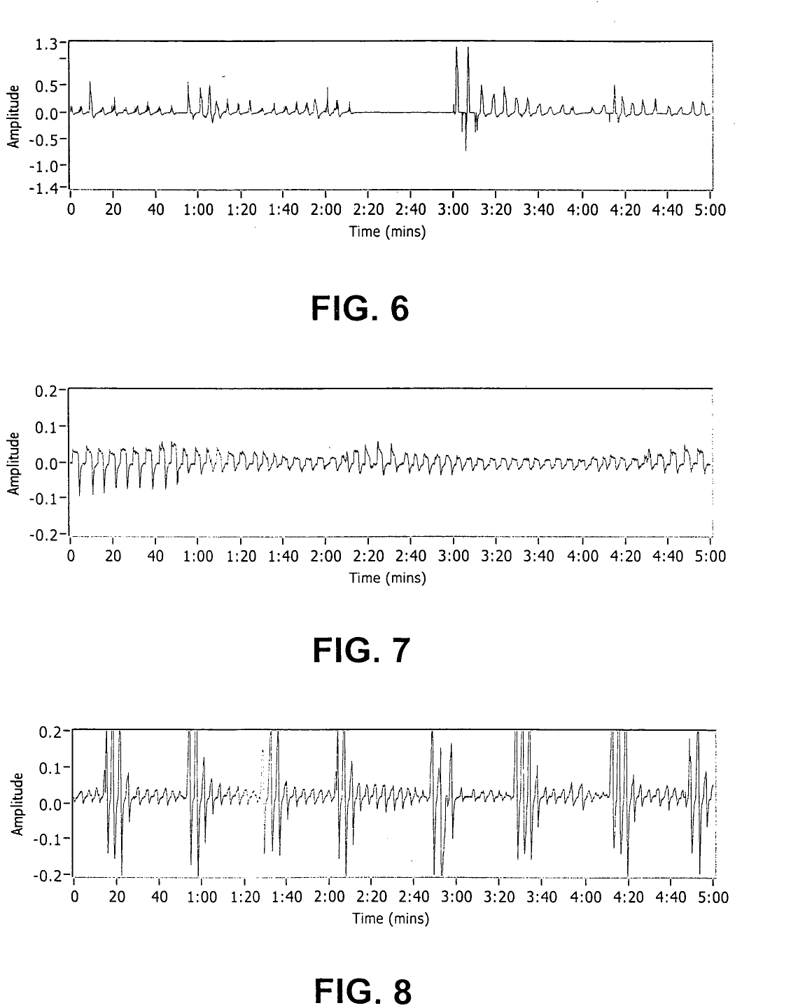

Sleep apnea may be detected with reference to the amplitude of the pressure signals received. By way of example, where the amplitude of the pressure signal is less than approximately 20% of that patient's normal breathing magnitude for a period greater than 10 seconds, a sleep apnea event can be identified as having occurred. An example of such an event is identified in FIG. 6, which comprises a plot of stored breath pressure (vertical axis) versus time (horizontal axis) that was captured using the data acquisition unit 104.

Upper Airway Resistance Syndrome (UARS) Detection

Upper airway resistant syndrome (UARS) can be identified, for example, if the amplitude of the patient's breathing signal is determined to form a generally square wave pattern (either during inhalation or exhalation). An example of this phenomenon is illustrated in FIG. 7. Such a pattern of breathing may be detected using one or more curve-fitting algorithms.

Respiratory Effort Related Arousal (RERA) Detection

Respiratory effort related arousal (RERA) can be identified through detection of intermittent periods of high-magnitude pressure signals separated by relatively normal breathing periods (i.e., lower-pressure signals). An example of this phenomenon is illustrated in FIG. 8. Again, such a pattern of breathing may be detected using an appropriate mathematical algorithm. Such an algorithm can, for instance, be configured to seek breathing magnitudes that exceed that patient's normal breathing magnitudes by a given percentage (e.g., 50-100%) for a given period of time (e.g., more than 5 seconds).

Snoring Detection

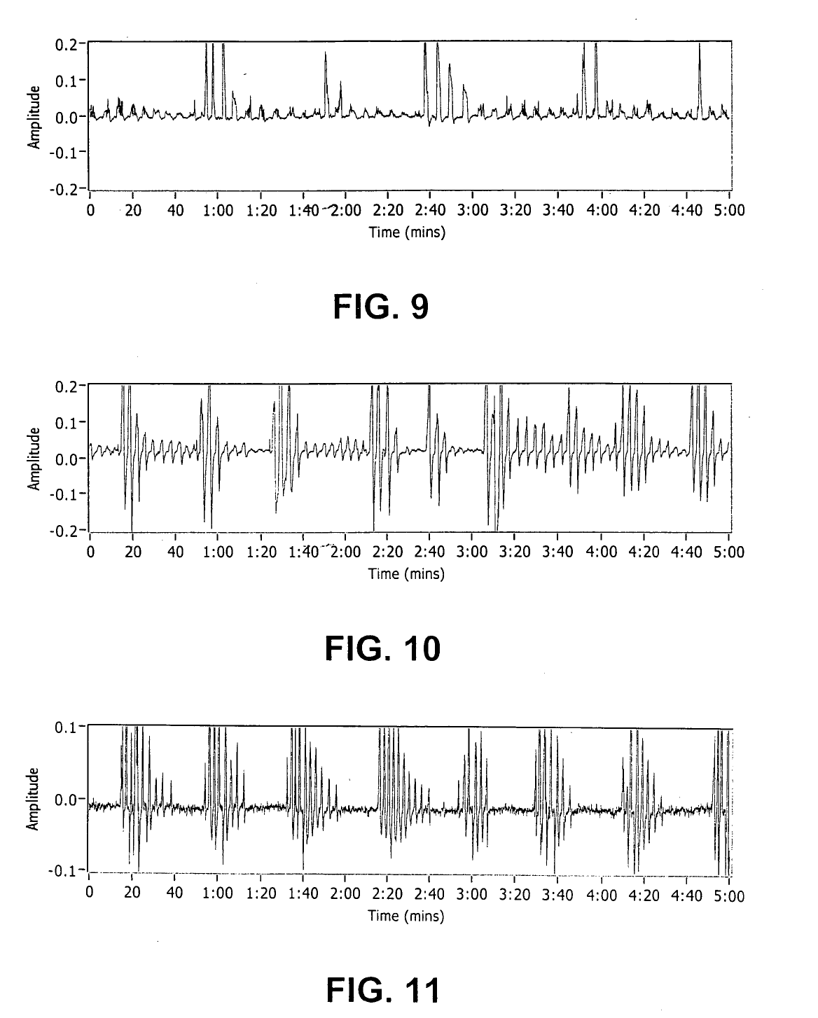

Snoring can be identified by detecting high-frequency (e.g., several spikes a second) spikes of pressure. An example of such high-frequency spikes is provided in FIG. 9. Once more, a mathematical algorithm can be used to make this determination. For instance, the algorithm can be configured to recognize a large number of pressure spikes or peaks (e.g., more than 5) in a relatively short period of time (e.g., 5 seconds).

Hypopnea Detection

Hypopnea can be identified, for example, where the amplitude of the pressure signal is less than approximately 50% to 70% of that patient's normal breathing magnitude for a given period, for instance, greater than 10 seconds. An example of such an event is identified in FIG. 10.

Cheyne-Stokes Breathing Detection

Cheyne-Stokes breathing can be identified, for instance, by detecting intermittent periods of high-magnitude pressure signals that are both separated by low-pressure signal periods and which are marked by signals that wax and slowly wane (i.e., attenuate). After the attenuation, apnea typically follows. An example of this phenomenon is illustrated in FIG. 11. Again, such a pattern of breathing may be detected using one or more appropriate mathematical algorithms. A suitable algorithm can, for instance, be configured to seek breathing magnitudes that exceed that patient's normal breathing magnitudes by a given percentage (e.g., 50-100%) for a given period of time (e.g., more than 5 seconds), and which slowly reduce in magnitude during that time period. Furthermore, the apnea detection algorithm described above can be used to detect any apnea events that occur after the signal waning.

Returning now to decision block 518 of FIG. 5B, if no sleep disordered breathing is identified, flow continues down to decision block 522 discussed below. If one or more sleep disordered breathing events are detected, however, flow continues to block 520 at which the sleep disordered breathing event occurrence is recorded in data acquisition unit memory 210. By recording these events in this manner, a diagnosing physician need not personally analyze the collected data to identify the patient's sleep disordered breathing, or can analyze the data in view of the automatically determined events.

At this point, it can be determined whether the underlying, collected data is to be saved, as indicated in decision block 522. This determination can, for example, be made in view of an operating mode that has been selected by the physician. If these data are not to be saved, i.e., only the sleep disordered breathing event data, if any, are to be stored (block 520), flow continues to block 524 and the underlying pressure and associated time data are deleted (or marked for deletion) from memory 210. If, on the other hand, these data are to be retained, for instance for the purpose of conducting more analysis and/or for providing a printout of the pressure data as a function of time, flow continues to decision block 526 at which it is determined whether the amount of data are to be reduced. If no such reduction of data is to be conducted, i.e., all collected pressure and time data are to be retained, flow continues down to decision block 532 described below. If reduction is to be performed, however, flow continues to block 528 at which data reduction is performed. This reduction can be performed in similar manner to that described above in relation to block 512 described above. Once this reduction has been performed, flow continues to block 530 and the remaining data are stored in the storage memory 210.

With reference next to decision block 532, it is determined whether there is time remaining in the data acquisition session. If time remains, flow returns to block 504 of FIG. 5A and pressure signals are continued to be received, stored, and analyzed in the manner described above. If not, however, flow for the session is terminated.

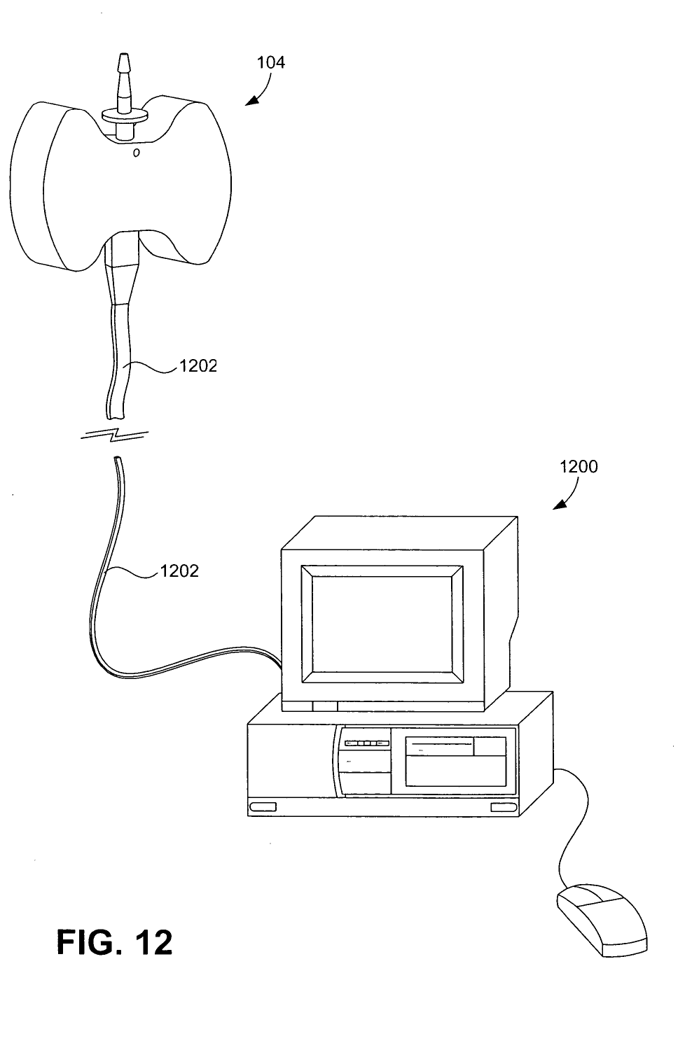

As noted above, data stored in the data acquisition unit 104 may be transmitted to another device for further analysis or output as a report. Such a situation is depicted in FIG. 12. As is shown in that figure, the data acquisition unit 104 can connect to a computer 1200, such as a PC, with an appropriate cable 1202, such as a USB cable. Although a cable connection is shown, data transmission could be effected wirelessly where the data acquisition unit 104 and the computer 1200 are equipped with appropriate wireless transmission components.

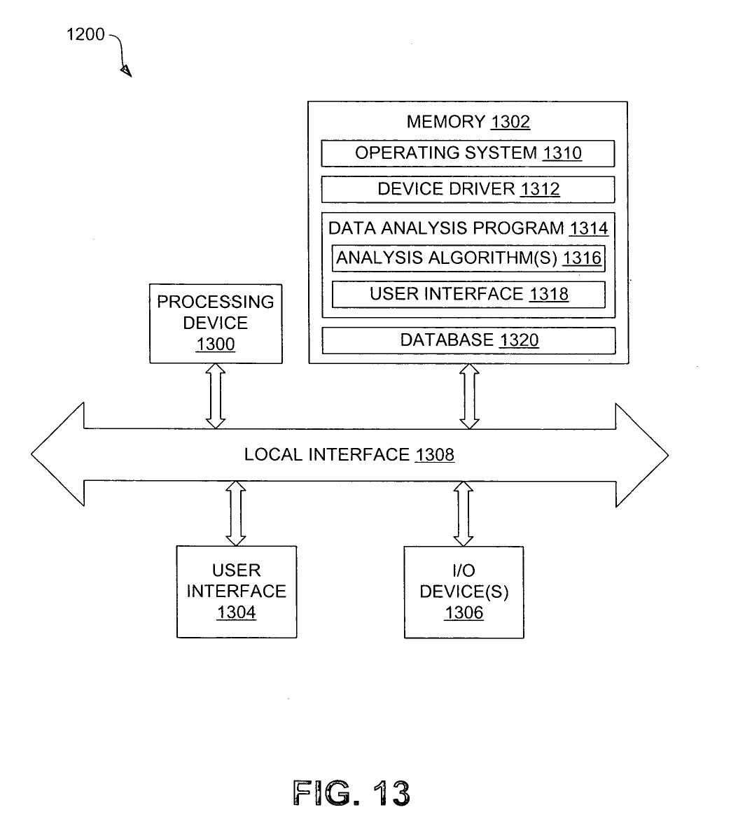

FIG. 13 is a block diagram that illustrates an example embodiment for the computer 1200. As indicated in FIG. 13, the computer 1200 comprises a processing device 1300, memory 1302, a user interface 1304, and at least one input/output (I/O) device 1306, each of which is connected to a local interface 1308.

The processing device 1300 can include a central processing unit (CPU) or an auxiliary processor among several processors associated with the computer 1200, or a semiconductor based microprocessor (in the form of a microchip). The memory 1302, includes any one of or a combination of volatile memory elements (e.g., RAM) and nonvolatile memory elements (e.g., hard disk, read only memory (ROM), etc.).

The user interface 1304 comprises the components with which a user interacts with the computer 1200. The user interface 1304 may comprise, for example, a keyboard, mouse, and a display. The one or more I/O devices 1306 are adapted to facilitate communications with other devices and may include one or more of a USB or a small computer system interface (SCSI) connection component.

The memory 1302 comprises various programs including an operating system (O/S) 1310, a device driver 1312, and a data analysis program 1314. The O/S 1310 controls the execution of other programs and provides scheduling, input-output control, file and data management, memory management, and communication control and related services. The device driver 1312 comprises a program that is used to communicate with the data acquisition unit 104 and, therefore, controls downloading of data collected from that device to the computer 1200.

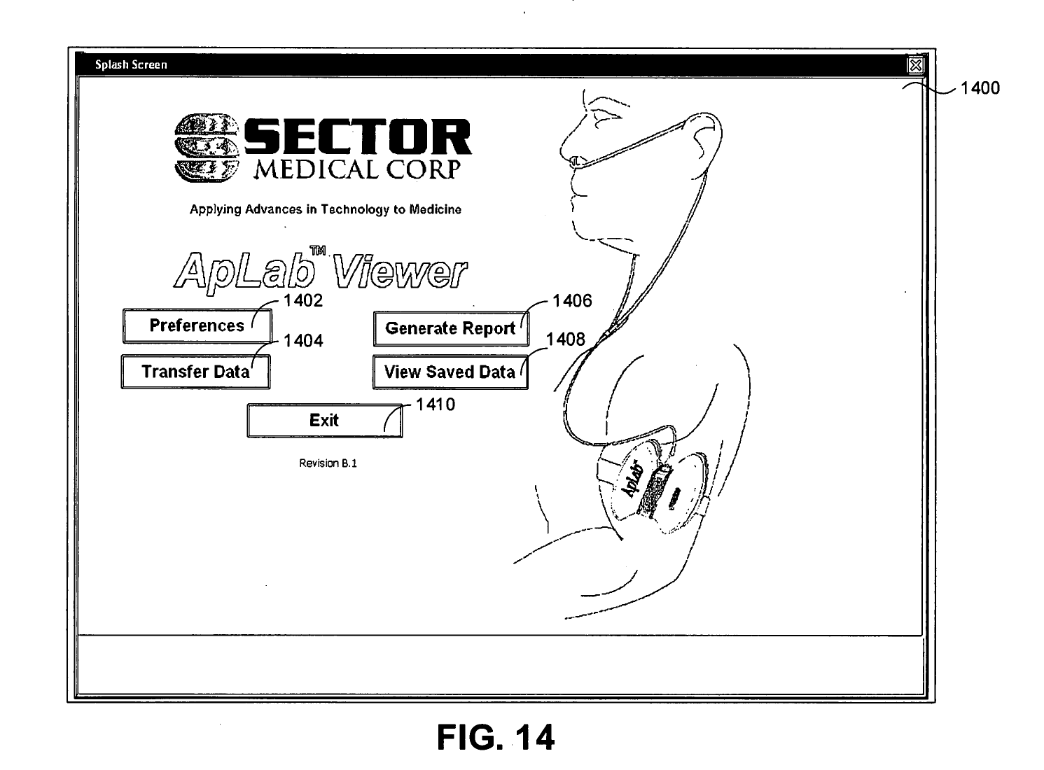

The data analysis program 1314 is used to analyze, display, and/or output the data downloaded to the computer 1200 (e.g., to the database 1320) from the data acquisition unit 104. As is indicated in FIG. 13, the program 1314 comprises, by way of example, one or more data analysis algorithms 1316, and a user interface 1318. The data analysis algorithms 1316 may be similar in function to those described above in relation to the data acquisition unit 104. Normally, however, the algorithms 1316 are specifically configured to format and organize the collected data, and the determinations gleaned therefrom, for the user interface 1318. As is apparent from FIGS. 14-22 below, the user interface 1318 comprises instructions used to support various interface screens that may be presented to a user, such as a physician.

Reference is next made to the interface screens shown in FIGS. 14-22 to explain use and operation of the data analysis program 1314 identified in FIG. 13. For the purposes of the following discussions, it is presumed that raw data collected by the data acquisition unit 104 has been downloaded to the computer 1200. Such downloading can be accomplished, for example, by connecting (either via a cable or wirelessly) with the data acquisition unit 104 and selecting an appropriate download command using the device driver 1312 (FIG. 13), which may be separate from the data analysis program 1314. In such a case, raw data has been stored to memory (e.g., a hard disk) of the computer 1200 and is available for manipulation using the data analysis program 1314.

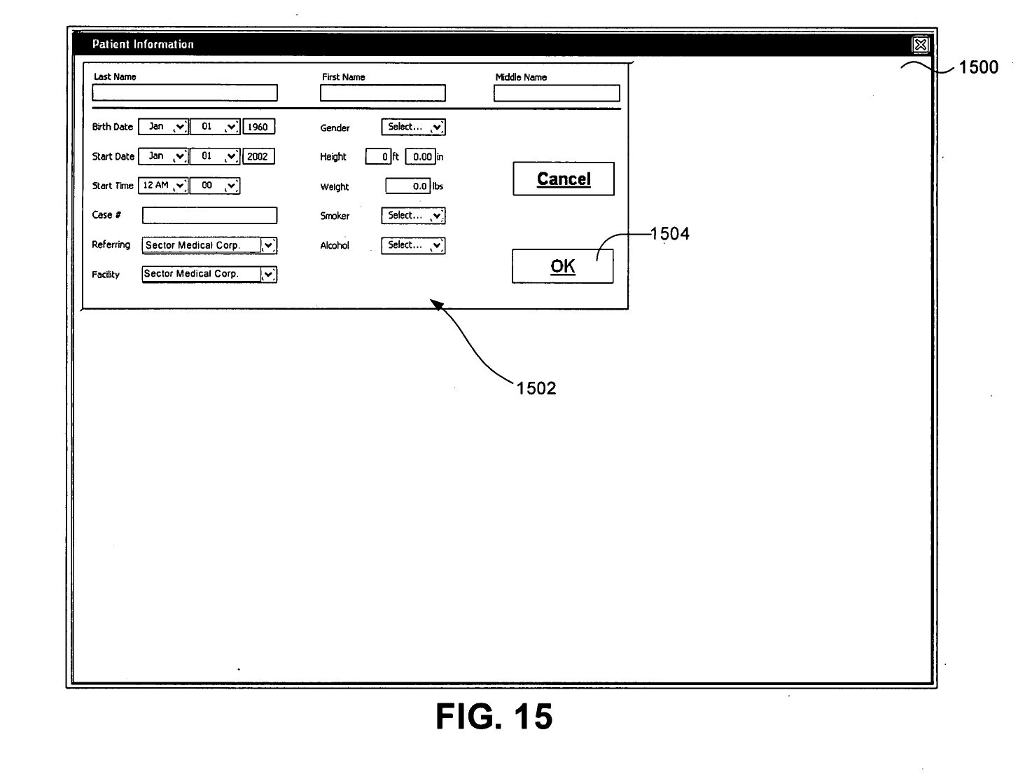

Beginning with FIG. 14, shown is an example initial interface screen 1400 that is presented to the user when the program 1314 is first activated, for example by selecting an appropriate icon from the computer desktop or program list. As is shown in FIG. 14, the interface screen 1400 includes several selectable “buttons” including a Preferences button 1402, a Transfer Data button 1404, a Generate Report button 1406, a View Saved Data button 1408, and an Exit button 1410. Assuming, as noted above, that raw data collected by the data acquisition unit 104 has already been downloaded to the computer 1200, that data may be accessed using the Transfer Data button 1404. Specifically, when that button 1404 is selected, a Patient Information screen 1500 shown in FIG. 15 is presented to the user. With reference to FIG. 15, the Patient Information screen 1500 comprises various data fields 1502 in which patient information, physician information, and sleep session information may be entered and/or selected. In regard to patient information, information can be provided about the patient whose data is going to be accessed including the patient name, birth date, gender, height and weight, and the patient's habits with respect to smoking and alcohol usage. Regarding information about the sleeping session, information can be provided about the case number, and the start date and time when the sleep session occurred (i.e., the duration in which pressure and time data was collected). With regard to the physician information, information can be provided regarding the referring physician and the physician facility. The above information will be added as header information to a new file that will be created using the data collected for the identified patient.

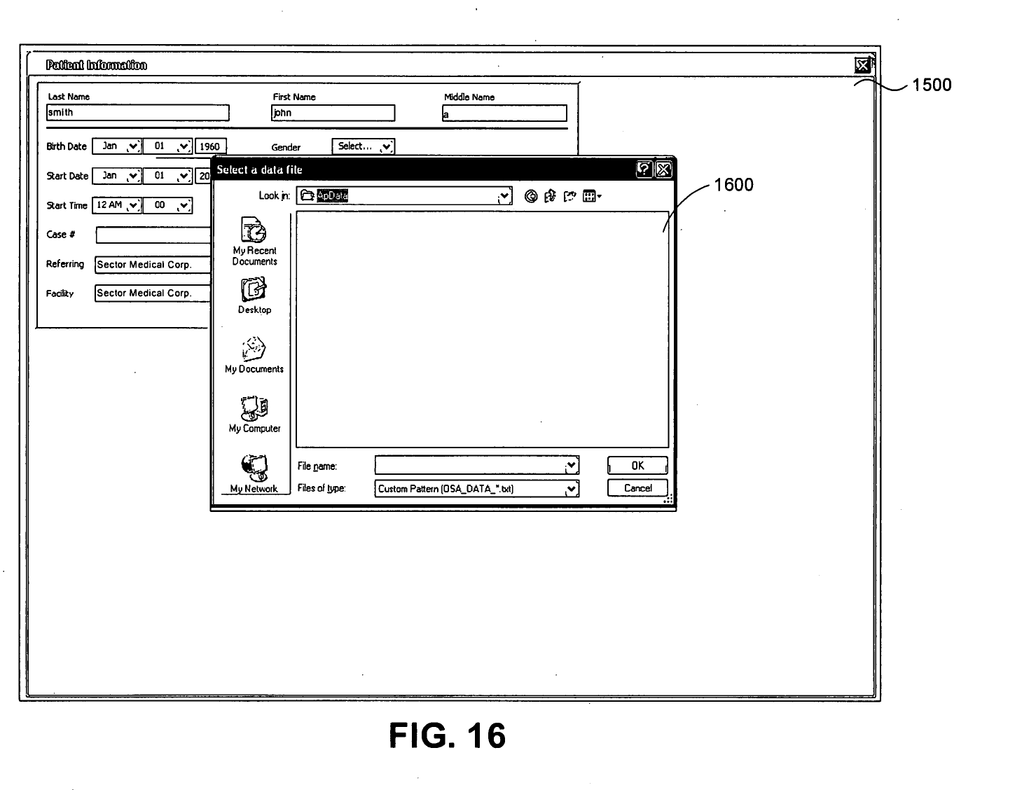

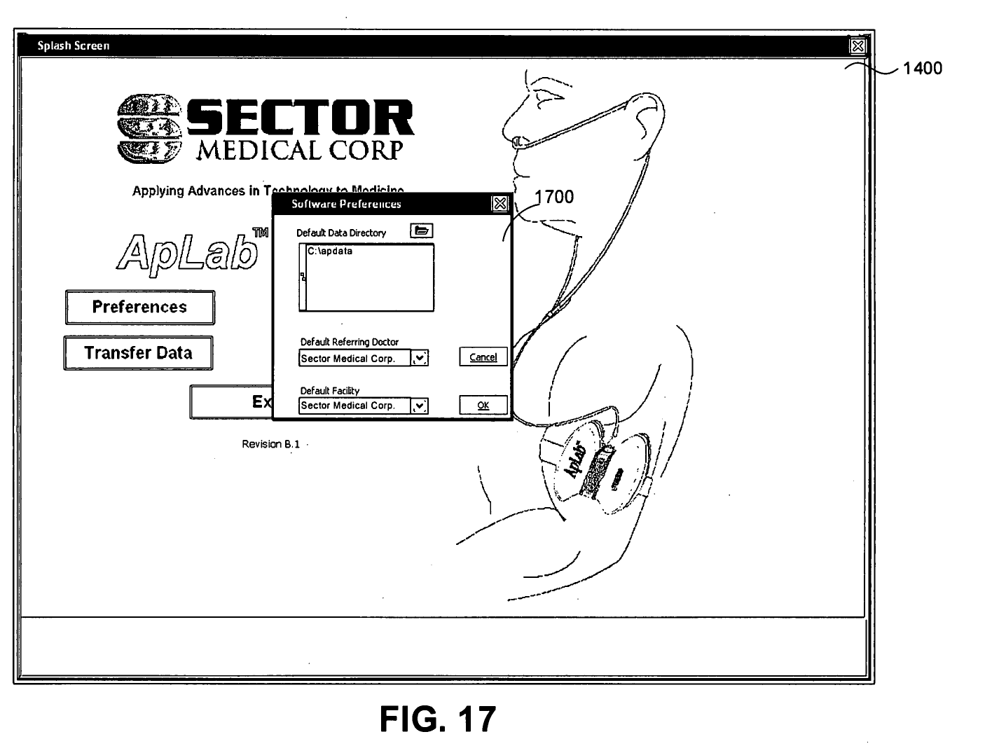

Once all relevant information has been entered into the data fields 1502, the user may select the OK button 1504. Upon selection of that button 1504, a Select a data file window 1600 shown in FIG. 16 is presented to the user. With reference to FIG. 16, the Select a data file window 1600 provides the user with the opportunity to select the file that has been downloaded to the computer 1200 and that contains the raw data that was collected in the sleep session at issue. In particular, the user can browse through computer memory (e.g., hard disk) to locate the file that was created as a result of the data download. Once the selection has been made, a new file is created and stored in computer memory (e.g., hard disk) that, as described above, comprises the raw data and the header information. Notably, the Select a data file window 1600 can, by default, identify a folder or directory that has been preselected by the user. Such preselection may be effected through selection of the Preferences button 1402 of the initial interface screen 1400 shown in FIG. 14. When that button 1402 is selected, the user is presented with a Software Preferences window 1700 shown in FIG. 17. That window 1700 permits the user to select a default data directory, a default referring doctor, and a default facility that will be used by default when a new file is created using the process described above with reference FIGS. 15 and 16. The default data directory may be selected by browsing the computer memory (e.g., hard disk), and the default referring doctor and default facility may be selected from drop down menus. Optionally, the available referring doctor and facility choices of the drop down menus are generated using a creation functionality provided by the Patient Information screen 1500 of FIG. 15.

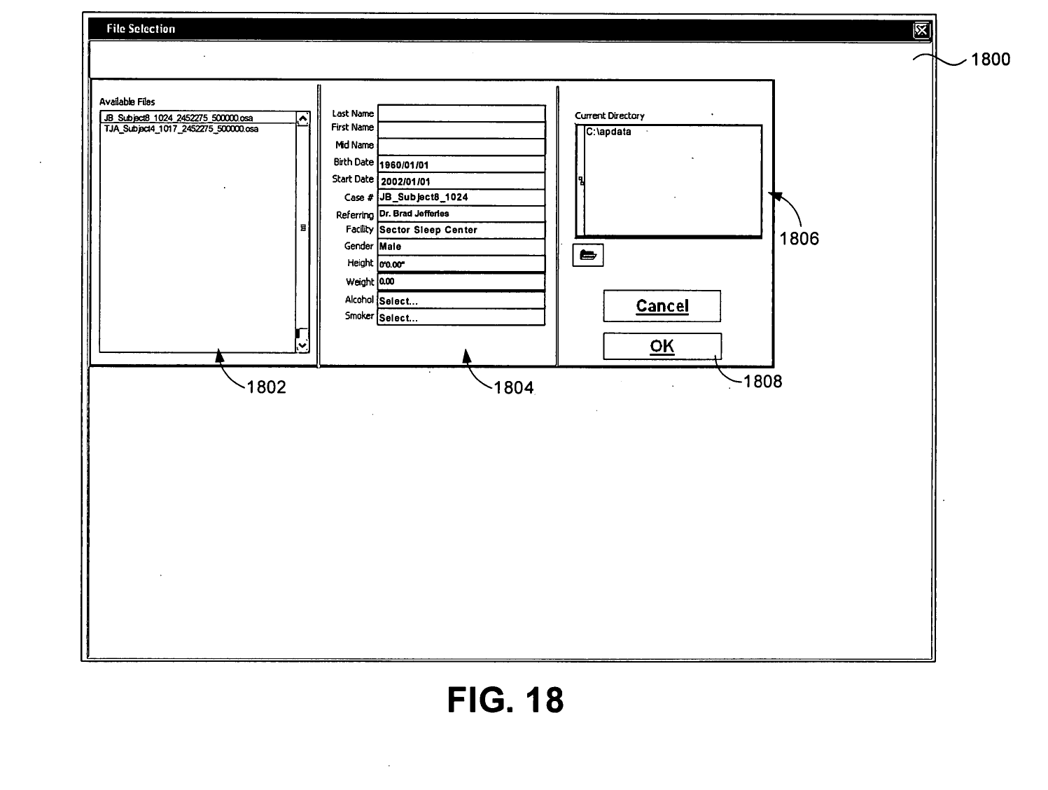

At this point, a file exists for the patient sleep session that comprises the data collected during the session and the various header information collected using the Patient Information screen 1500 of FIG. 15. The user may now generate a hard copy report and/or view the collected data, as well as analysis of that data, using the data analysis program 1314. To generate a report, the user selects the Generate Report button 1406 of the initial interface screen 1400 shown in FIG. 14. When that button 1406 is selected, a File Selection screen 1800 shown in FIG. 18 is presented to the user as indicated in FIG. 18. As shown in that figure, the File Selection screen 1800 comprises an Available Files field 1802 that identifies all of the files that have been created (using the process described above) in association with a given directory identified in a Current Directory field 1806. In the example of FIG. 18, a file entitled “JD_Subject8_{—}1024_{—}2452275_{—}500000.osa” is highlighted. When a file is so highlighted, the header information that was collected using the Patient Information screen 1500 is shown in various fields 1804 of the File Selection screen 1800. These fields 1804 aid the user in selecting the desired file in situations in which the user does not recognize the desired file from its file name alone.

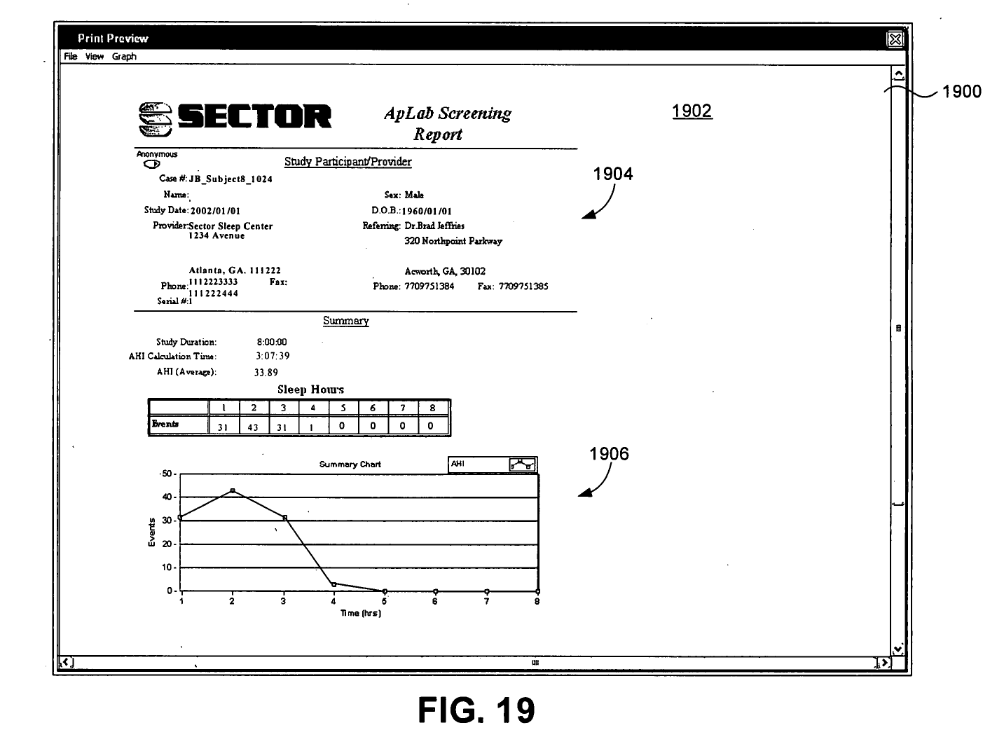

Once the desired file has been identified, the user can select the OK button 1808 to open the file. When the OK button 1808 is selected, the highlighted file is accessed and a report 1902 suitable for printing is presented to the user in a Print Preview screen 1900 shown in FIG. 19. Referring to FIG. 19, the report 1902 comprises the various header information 1904 (e.g., patient information, physician information, and sleep session information), as well as multiple plots of the observed pressures as functions of time (not visible in FIG. 19). Those plots comprise time snapshots, for instance in five minute increments, of the total sleep session and present the various pressure information in graphical form. Visible in FIG. 19 is an events plot 1906 that summarizes the frequency of various sleep disordered breathing (SDB) events identified by the data analysis program 1314 over the course of the sleep session. In the example of FIG. 19, only three hours of the available eight hour session were used to collect data (thus explaining the absence of events in hours 4-8). The full report 1902 can be printed using an appropriate print process such as a “File, Print” process well known in window-type program usage.

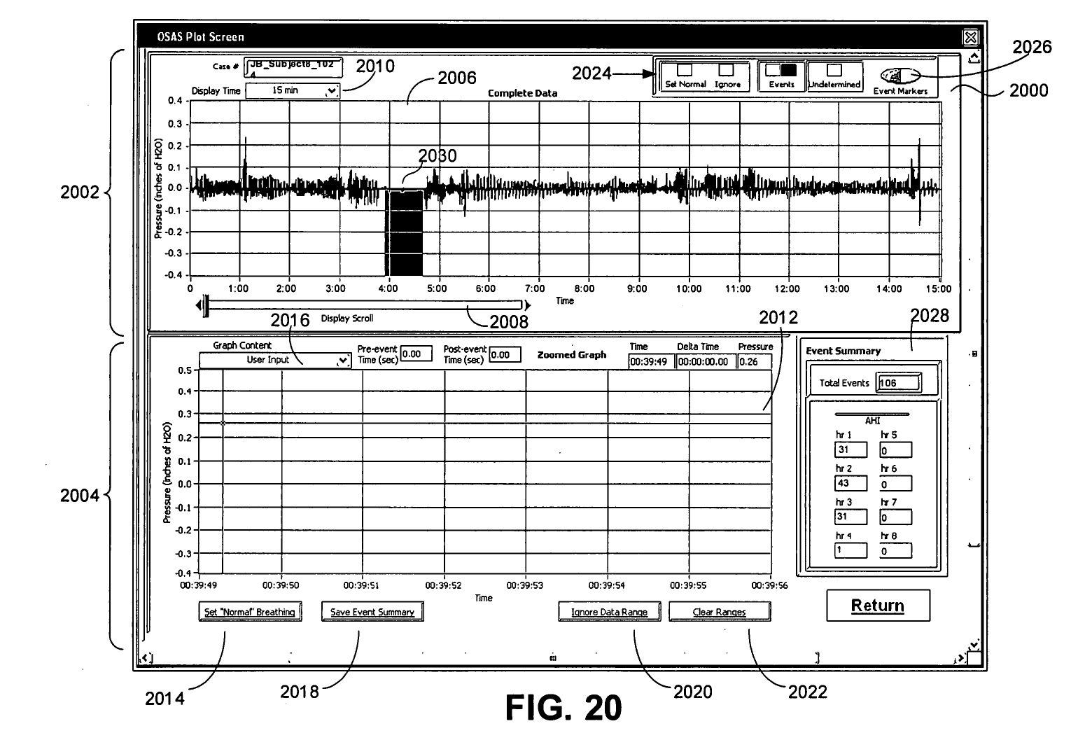

Referring back to FIG. 14, the collected data and the various conclusions (e.g., identified SDB events) can be reviewed by selecting the View Saved Data button 1408. When that button 1408 is selected, the user is presented with a File Selection screen similar to that described above in relation to FIG. 18. Through use of that screen, a desired file can be selected and, once so selected, an SDB Plot Screen 2000 shown in FIG. 20 is presented to the user. Referring now to FIG. 20, the SDB Plot Screen 2000 is generally divided into a first (e.g., top) portion 2002 and a second (e.g., bottom) portion 2004. The first portion 2002 contains a Complete Data graph 2006 that plots pressure in inches of water (vertical axis) against time in minutes (horizontal axis) for the entire sleep session. Of the total sleep session, time 0 to time 15:00 is visible in FIG. 20. The other time periods are viewable, however, through manipulation of a Display Scroll bar 2008. The time increments visible at any given time are selectable using a drop down menu 2010 provided adjacent the graph 2006. In addition to the plot of pressure versus time, indicated in the Complete Data graph 2006 are time periods in which a determined SDB event has occurred. As is described in greater detail below, the various SDB events can be identified using different colors.

The second portion 2004 of the screen 2000 contains a Zoomed Graph 2012 that can be used to highlight, or zoom in on, a particular time period of interest identified in the Complete data graph 2006. The process in which that is achieved is described below. In addition to the Zoomed Graph 2012, the second portion 2004 includes a Set “Normal” Breathing button 2014 that the user can use to designate a given time period of the Complete Data graph 2006 that is deemed to reflect normal, i.e., non-SDB breathing of the patient. In particular, when that button 2014 is selected, the user can highlight a given time period within the Complete Data graph 2006 that is believed to reflect a period of normal breathing for the patient. Each identified SDB event is listed for the user in a drop-down menu 2016. When the menu is accessed, particular SDB events can be viewed in the Zoomed Graph 2012. All such events can be stored in a separate file, if desired, by selecting a Save Event Summary button 2018 also provided in the second portion 2004.

In situations in which a portion of the sleep session is to be ignored in the breathing analysis, for instance if data was only collected during a portion of the possible eight hour sleep session, the user can select the Ignore Data Range button 2020 to identify the time period that is to be ignored. Once the normal breathing and/or data range to be ignored have been set, those selections can be cleared, if desired, using the Clear Ranges button 2022.

As is further shown in FIG. 20, a color-coded key 2024 for the Complete Data graph 2006 is provided in the first portion 2002 of the screen 2000. That key 2024 identifies the color scheme for color-coded bars that identify in the Complete Data graph 2006 the time periods pertaining to normal breathing time periods, time periods to be ignored, time periods that have been determined by the data analysis program 1314 to contain an SDB event, and time periods that do not fit the rubrics of known SDB events but nonetheless are abnormal or atypical in some way (i.e., “undetermined” events). Optionally, the normal breathing time periods and the time periods to be ignored are identified by color bars positioned above the zero pressure line in the Complete Data graph 2006, and the time periods that have been determined to contain an SDB event or an undetermined event are represented with color bars positioned below the zero pressure line in that graph. One example of such a bar or marker is shown in FIG. 20 (reference numeral 2030). All such bar, or markers, can be removed from view (or returned to view) using the Event Markers button 2026.

With further reference to FIG. 20, the second portion 2004 of the screen 2000 further includes an Event Summary 2028 that provides a summary of the various determined SDB events. As indicated in FIG. 20, the Event Summary 2028 identifies the total number of SDB events identified as well as the number of SDB events that occurred during each individual hour of the sleep session.

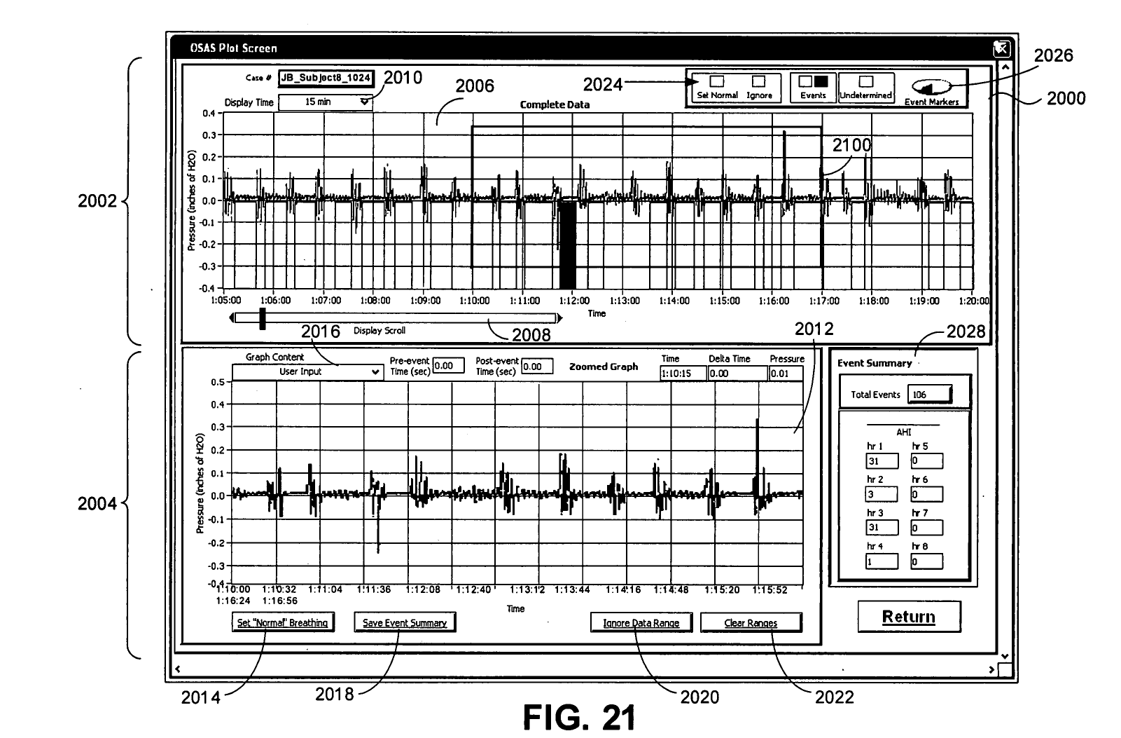

Turning to FIG. 21, the Complete Data graph 2006 has been scrolled to a new time period in which various colored bars (markers) are shown below the zero pressure line. These bars identify one apnea event (dark bar) and several hypopnia events (light bars) that have been identified by the data analysis program 1314. As is further indicated in the Complete Data graph 2006, a portion of the viewable sleep session has been highlighted using a selection box 2100 generated using a drag-and-release process with an appropriate user interface device, such as a mouse. Upon such highlighting, the selected time period is shown in greater detail (i.e., expanded) in the Zoomed Graph 2012.

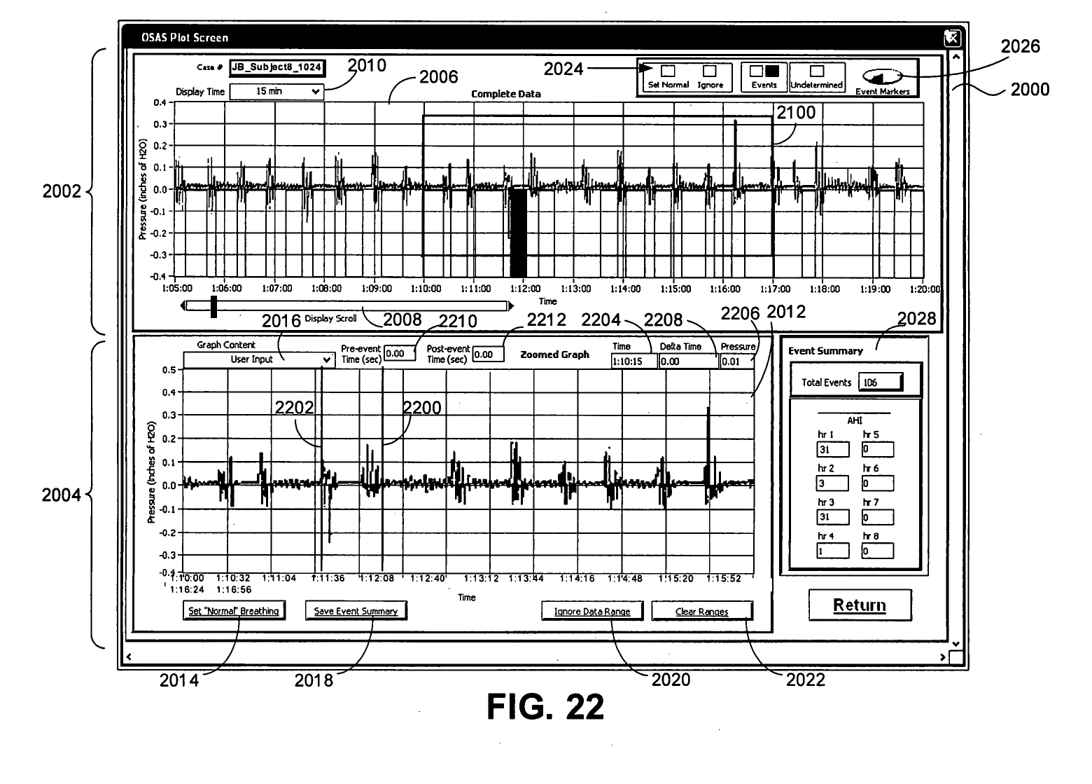

Referring next to FIG. 22, illustrated is manipulation of the data contained in the Zoomed Graph 2012. As shown in FIG. 22, a first vertical line 2200 has been positioned along the time line of the graph 2012, for instance using a mouse, as has a second vertical line 2202, to identify the bounds of a further selected time period. The position of one of the vertical lines 2200, 2202 (depending upon which is used for that purpose as indicated by, for example, a particular line color) in terms of time and pressure is indicated in a Time field 2204 and a Pressure field 2206, respectively. In addition, the amount of time between the two lines 2200, 2202 is identified in a Delta Time field 2208. If desired, the further selected time period may be expanded beyond the selected bounds using a Post-event Time field 2210 and a Post-event Time field 2212. In particular, time periods in seconds may be designated in those fields 2210, 2212 to expand the further selected time period by those amounts.

With the various interface tools described above in relation to FIGS. 14-22, the user, for example a physician, can scrutinize the data collected by the data acquisition unit 104 and the SDB event determinations made by the data analysis program 1314 to analyze the patient's sleeping patterns to make a diagnosis and, potentially, determine an appropriate treatment plan. In addition, the user can focus in on particular breathing events, whether they be SDB events or other events, using those tools.

Various programs algorithms (i.e. logic) have been described herein. These programs can be stored on any computer-readable medium for use by or in connection with any computer-related system or method. In the context of this document, a computer-readable medium is an electronic, magnetic, optical, or other physical device or means that contains or stores a computer program for use by or in connection with a computer-related system or method. These programs can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

1. A portable data acquisition unit, comprising:
a pressure sensor that is configured to measure pressure signals at a high-frequency rate, the signals pertaining to patient breathing;
a microcontroller that receives pressure signals measured by the pressure sensor and determines clocks times associated with the pressure signals; and
an interface that is configured to output sleep session data from the data acquisition unit to another device.

2. The portable data acquisition unit of claim 1, wherein the pressure sensor comprises a solid-state pressure sensor.

3. The portable data acquisition unit of claim 2, wherein the solid-state pressure sensor comprises strain gauges that measure pressure.

4. The portable data acquisition unit of claim 2, wherein the solid-state pressure sensor comprises a differential bridge, true low-pressure, silicon die sensor.

5. The portable data acquisition unit of claim 1, wherein the pressure sensor is capable of measuring 6,000 to 10,000 pressure signals per second.

6. The portable data acquisition unit of claim 1, wherein the microcontroller is a digital signal processor.

7. The portable data acquisition unit of claim 1, wherein the microcontroller comprises non-volatile memory.

8. The portable data acquisition unit of claim 7, wherein the non-volatile memory comprises logic that controls generation of sleep session data.

9. The portable data acquisition unit of claim 7, wherein the non-volatile memory comprises logic that analyzes the pressure signals and the clocked times to identify sleep disordered breathing events.

10. The portable data acquisition unit of claim 1, wherein the interface comprises a universal serial bus interface.

11. The portable data acquisition unit of claim 1, further comprising a battery that powers the data acquisition unit.

12. The portable data acquisition unit of claim 1, further comprising an amplifier that amplifies the pressure signals measured by the pressure sensor and an analog-to-digital converter that converts the amplified pressure signals into digital signals for the microcontroller.

13. A portable data acquisition unit configured to collect patient information during a sleep session, the unit comprising:
a housing that is configured to be attached to a patient or clothing of the patient;
a solid-state pressure sensor that is configured to measure pressure signals collected by a patient interface positioned adjacent nostrils of the patient;
an amplifier that amplifies the pressure signals measured by the pressure sensor;
an analog-to-digital converter that converts the amplified pressure signals into digital signals;
a microprocessor that receives the digital signals from the analog-to-digital converter and determines clocks times associated with the digital signals;
a battery that powers the data acquisition unit; and
an interface that is configured to transfer pressure and time data from the data acquisition unit to another device.

14. The portable data acquisition unit of claim 13, wherein the housing is configured to mount to an arm of the patient using an arm band.

15. The portable data acquisition unit of claim 13, wherein the solid-state pressure sensor comprises a differential bridge, true low-pressure, silicon die sensor.

16. The portable data acquisition unit of claim 13, wherein the pressure sensor is capable of measuring 6,000 to 10,000 pressure signals per second.

17. The portable data acquisition unit of claim 13, wherein the microcontroller is a digital signal processor that comprises non-volatile memory that includes logic that controls generation of sleep session data.

18. The portable data acquisition unit of claim 17, wherein the non-volatile memory further includes an algorithm that is configured to analyze pressure and time data to identify sleep disordered breathing events.

19. The portable data acquisition unit of claim 18, wherein the microcontroller is configured to mark identified sleep disordered breathing events to identify them to a user.

20. The portable data acquisition unit of claim 13, wherein the interface comprises a universal serial bus interface.

21. A data acquisition program stored on a computer-readable medium, the program comprising:
logic configured to receive measured pressure signals collected at a high-frequency rate and store the signals as pressure data;
logic configured to determine times at which the measured pressure signals were measured and store the times as time data;
logic configured to transmit the pressure and time data to a computer.

22. The program of claim 21, further comprising logic configured to reduce the amount of stored data.

23. The program of claim 21, further comprising logic configured to compress the stored data.

24. The program of claim 21, further comprising logic configured to identify sleep disordered breathing events through analysis of the pressure data and the time data.

25. The program of claim 21, wherein the program is stored on a medium of a portable data acquisition device.

26. A method of collecting sleep session data for the purpose of identifying sleep disordered breathing of a patient, the method comprising:
providing a portable data acquisition unit to the patient, the data acquisition unit being configured to collect pressure data at a high-frequency rate;
measuring pressure signals that pertain to patient breathing with the data acquisition unit at a high-frequency rate as the patient sleeps and recording a time at which each pressure signal is collected;
downloading pressure data and time data from the data acquisition unit to a computer; and
manipulating the downloaded data with the computer.

27. The method of claim 26, wherein providing a portable data acquisition unit to the patient comprises providing a portable data acquisition unit to the patient that is configured for wearing by the patient as the patient sleeps.

28. The method of claim 26, wherein collecting pressure signals with the data acquisition unit at a high-frequency rate comprises collecting pressure signals at a rate of 6,000 to 10,000 pressure signals per second.

29. The method of claim 26, wherein manipulating the downloaded data with a computer comprises printing the downloaded data as a report.

30. The method of claim 26, wherein manipulating the downloaded data displaying presenting plots of pressure versus time.

31. The method of claim 26, further comprising analyzing the pressure data and the time data to identify sleep disordered breathing events.

32. The method of claim 31, wherein analyzing the pressure data and the time data comprises analyzing the data with the data acquisition unit prior to downloading.

33. The method of claim 31, wherein analyzing the pressure data and the time data comprises analyzing the data with the computer after downloading.

34. A data analysis program stored on a computer-readable medium, the program comprising:
logic configured to receive pressure and time data collected by a portable data acquisition unit during a patient sleep session;
logic configured to analyze the pressure and time data to determine sleep disordered breathing events; and
logic configured to generate a graph that plots pressure versus time, the graph including markers that identify the determined sleep disordered breathing events.

35. The program of claim 34, further comprising logic configured to collect user-entered data regarding the patient and append that data to the pressure and time data to create a sleep session file.

36. The program of claim 34, further comprising logic configured to assemble the pressure and time data and generate a printable report that comprises at least one graph that plots pressure versus time.

37. The program of claim 34, further comprising logic configured to display a complete data graph that plots pressure and time data for the entire sleep session.

38. The program of claim 37, further comprising logic configured to designate a selected time period of the complete data graph as a normal breathing time period.

39. The program of claim 37, further comprising logic configured to present a zoomed graph that comprises an expanded view of a portion of the data of the complete data graph.

40. The program of claim 39, further comprising logic configured to enable designation of start and end points that define a portion of time of the zoomed graph.

41. A user interface for presenting sleep session data to a user, the interface comprising:
a patient information screen with which information concerning a patient whose breathing was monitored during a sleep session using a portable data acquisition unit can be collected;
a select a data file screen with which a pre-existing file containing raw data collected during the sleep session can be selected for the purpose of creating a sleep session file that includes the raw data and the patient information; and
a sleep disordered breathing screen generated using the created sleep session file, the sleep disordered breathing screen displaying a plot of pressure data versus time data in a graph, the sleep disordered breathing screen further displaying markers in the graph that identify automatically-determined sleep disordered breathing events to the user.

42. The interface of claim 41, wherein different markers are used to identify different types of determined sleep disordered breathing events in the graph.

43. The interface of claim 41, wherein the sleep disordered breathing screen is capable of displaying in the graph a marker that identifies normal breathing of the patient.

44. The interface of claim 41, wherein the sleep disordered breathing screen is capable of displaying in the graph markers that identify undetermined events that are not recognized sleep disordered events but which relate to a typical patient breathing.

45. The interface of claim 41, wherein the sleep disordered breathing screen further comprises a zoomed graph in which a portion of data displayed in the other graph can be displayed in an expanded form.

46. The interface of claim 45, wherein the sleep disordered breathing screen further comprises tools that enable the user to conduct analysis of the data displayed in the zoomed graph.

47. The interface of claim 41, wherein the sleep disordered breathing screen further comprises an event summary area in which the total number of determined sleep disordered events and the total number of determined sleep disordered events per hour of the sleep session are displayed.
